(19) United States
(12) Patent Application Publication
(10) Pub. No.: US 2025/0218557 A1
(43) Pub. Date: Jul. 3, 2025

(54) METHOD FOR DISPLAYING MEDICAL INFORMATION BASED ON ALLOCATED RESOURCE

(71) Applicant: IRM Inc., Seoul (KR)

(72) Inventor: Seung Wook CHOI, Seongnam-si (KR)

(73) Assignee: IRM Inc., Seoul (KR)

(21) Appl. No.: 19/006,067

(22) Filed: Dec. 30, 2024

(30) Foreign Application Priority Data
Dec. 28, 2023 (KR) 10-2023-0193848
Feb. 28, 2024 (KR) 10-2024-0028448

(51) Int. Cl. G16H 10/60; G16H 15/00

(52) U.S. Cl. CPC G16H 10/60; G16H 15/00

(57) ABSTRACT

A method for displaying medical information based on the resources allocated to the medical information display includes calculating the amount of data that can be processed by the resources, identifying the medical information that needs to be processed by the resources according to the viewing mode requested by the user, and if the data amount of the identified medical information is greater than the calculated data amount, displaying the remaining medical information excluding some of the information.

### CROSS-REFERENCE TO RELATED APPLICATION

The present application is based upon and claims the benefit of priority to Korean Patent Application No. 10-2023-0193848, filed on Dec. 28, 2023, and Korean Patent Application No. 10-2024-0028448, filed on Feb. 28, 2024. The disclosures of the above-listed applications are hereby incorporated by reference herein in their entirety.

### TECHNICAL FIELD

The technical idea of the present disclosure relates to a method of displaying medical information, and more specifically, to a method of displaying medical information based on an allocated resource.

### BACKGROUND TECHNOLOGY

In the past, patients' medical information was managed by writing it on paper, but now it has been computerized in the form of electronic medical records. Hospitals have systems constructed to view, record, and utilize the electronic medical records, and users access the electronic medical records through user terminals, such as computers, to view and record the electronic medical records.

However, the interface of the existing system for viewing and entering electronic medical records is structured in the form of a library, which has a drawback of making the procedure for accessing desired records complicated. In addition, since medical information is displayed through a separate window based on date or type of the medical information, it is difficult for users to check chronological changes in the records and perform treatment based on the checked result. The traditional system interface is complex, resulting in limited access and usage for users, such as patient without expertise, and has low applicability to user terminals that are diversifying, such as tablet PCs and mobile phones.

### SUBJECT MATTER OF THE INVENTION

### Problem to be Solved

The present invention is directed to providing a method of differentially displaying medical information based on the performance of an allocated resource.

### Means for Solving the Problem

According to an aspect of the present invention, there is provided a method of displaying medical information based on an allocated resource, which includes: producing a data amount that is processable by the resource; specifying medical information to be rendered or processed by the resource according to a viewing mode requested by a user; and filtering some information of the medical information to display remaining medical information when the data amount of the specified medical information is larger than the computed data amount.

The viewing mode may be divided according to a range of a process accessible by the resource, and the data amount of the specified medical information increases according to the range of the process.

The displaying may include, when the medical information is generated as a storyboard in which the medical information is listed in time series, determining a time range of the storyboard to be filtered according to a difference between the data amount of the medical information and the computed data amount.

The displaying may include, when the medical information is an image including annotation information or a measurement value, displaying an image excluding the annotation information or the measurement value as a highest priority.

### EFFECTS OF THE INVENTION

The method for displaying medical information according to exemplary embodiments of this disclosure can differentially display medical information based on the resources of the device requesting the display of medical information. As a result, users can quickly acquire the necessary medical information through optimized means. The effects that can be obtained from the exemplary embodiments of this disclosure are not limited to the effects mentioned above. Other effects not mentioned can be clearly derived and understood by those skilled in the art from the description below. In other words, unintended effects that may arise from the implementation of the exemplary embodiments of this disclosure can also be derived by those skilled in the relevant technical field from the exemplary embodiments.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram illustrating an electronic device for displaying medical information according to an embodiment of the present disclosure;

FIG. 2 is a flowchart showing a method of displaying medical information according to an embodiment;

FIG. 3 is a diagram illustrating a storyboard in which treatment information is listed in time series according to an embodiment;

FIG. 4 is a diagram illustrating a medical image in which annotation information is described according to an embodiment; and

FIG. 5 is a diagram illustrating an example in which medical information is displayed on a different resource according to an embodiment.

### DETAILED DESCRIPTION OF EXEMPLARY EMBODIMENTS

Hereinafter, embodiments of the present specification will be described in detail with reference to the accompanying drawings. While embodiments according to the present disclosure are susceptible to various modifications and alternative forms, specific embodiments thereof are shown by way of embodiment in the drawings and will herein be described in detail. It should be understood, however, that there is no intent to limit the embodiments according to the concept of the present disclosure to the particular forms disclosed, but on the contrary, the embodiments according to the concept of the present disclosure are to cover all modifications, equivalents, and alternatives falling within the spirit and scope of the invention. Like numbers refer to like elements throughout the description of the drawings.

It will be further understood that the terms “comprise,” “comprising,” “include” and/or “including” used herein specify the presence of stated features, integers, steps, operations, elements, and/or components, but do not preclude the presence or addition of one or more other features, integers, steps, operations, elements, components, and/or groups thereof.

In various embodiments of the present disclosure, the expression “or” or the like includes any and all combinations of the items listed together in the corresponding phrase. For example, “A or B” may include A, may include B, or may include both A and B.

The expressions “first,” “second,” etc., used in various embodiments of the present disclosure may be used to describe various components of various embodiments, but do not limit the components. For example, the terms do not limit the sequence and/or importance of the components, and may be used to distinguish one component from another.

It will be understood that when an element is referred to as being “connected” or “coupled” to another element, the element can be directly connected or coupled to the other element or intervening elements may be present.

In the embodiments of the present disclosure, terms such as “module,” “unit,” “part,” etc., are terms used to refer to components that perform at least one function or operation, and these components may be implemented in hardware or software, or as a combination of hardware and software. In addition, a plurality of “modules,” “units,” “parts,” etc., may be integrated into at least one module or chip and implemented as at least one processor, except in cases in which each needs to be implemented as individual specific hardware.

It will be further understood that terms, such as those defined in commonly used dictionaries, should be interpreted as having meanings that are consistent with their meanings in the context of the relevant art and will not be interpreted in an idealized or overly formal sense unless expressly so defined herein.

Hereinafter, an embodiment of the present disclosure will be described in detail with reference to the attached drawings.

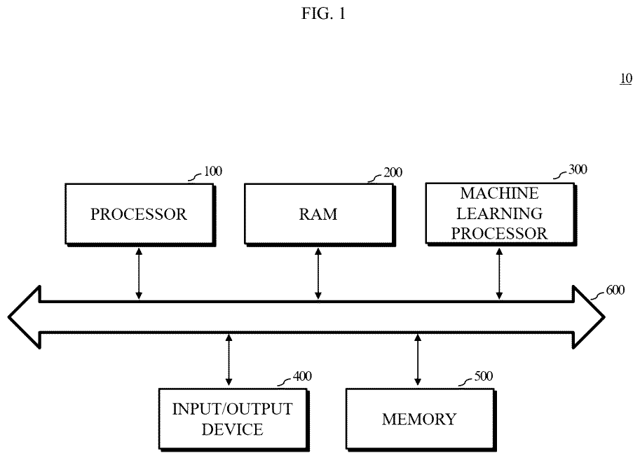

FIG. 1 is a block diagram illustrating an electronic device for displaying medical information according to an embodiment of the present disclosure.

Referring to FIG. 1, the electronic device 10 may differentially display medical information depending on the status of a resource requesting display of medical information. For example, the electronic device 10 may be applied to a computing device, a personal computer, a smart TV, a smart phone, a mobile device, a video display device, a measuring device, an Internet of things (IoT) device, etc., and may also be mounted on one of various types of electronic devices.

A resource may be referred to as a system resource in computing, and a system resource may be a component that includes limited physical components and virtual components within a computer system. That is, a resource may include all connected devices and internal system components, and a virtual system resource may include files (specifically, a file handle), a network connection (specifically, a network socket), and a memory area.

The resource may be connected to the electronic device 10, and the electronic device 10 may compare a data amount that is processable by the resource with a data amount of medical information by identifying a resource status, including a processor type and a network status of the resource.

The electronic device 10 may include at least one IP block and a machine learning processor 300. The electronic device 10 may include various types of IP blocks, and for example, as illustrated in FIG. 1, the electronic device 10 may include IP blocks such as a processor 100, a random access memory (RAM) 200, an input/output device 400, and a memory 500. In addition, the electronic device 10 may further include other general-purpose components such as a multi-format codec (MFC), a video module (e.g., a camera interface, a joint photographic experts group (JPEG) processor, a video processor, or a mixer), a 3D graphics core, an audio system, a display driver, a graphic processing unit (GPU), a digital signal processor (DSP), and the like.

Components of the electronic device 10, such as the processor 100, the RAM 200, the machine learning processor 300, the input/output device 400, and the memory 500, may transmit and receive data through a system bus 600. For example, as a standard bus specification, the advanced microcontroller bus architecture (AMBA) protocol of advanced RISC machine (ARM) may be applied to the system bus 600. However, it is not limited thereto, and various types of protocols may be applied.

In the embodiment, the components of the electronic device 10, the processor 100, the RAM 200, the machine learning processor 300, the input/output device 400, and the memory 500 may be implemented as a single semiconductor chip, and for example, the electronic device 10 may be implemented as a system on chip (SoC). However, it is not limited thereto, and the electronic device 10 may be implemented as a plurality of semiconductor chips. In an embodiment, the electronic device 10 may be implemented as an application processor mounted on a mobile device.

The processor 100 may control the overall operation of the electronic device 10, and as an example, the processor 100 may include at least one of a central processing unit (CPU) and a GPU. The processor 100 may include one core (single core) or multiple cores (multi-core). The processor 100 may process or execute programs and/or data stored in the RAM 200 and the memory 500. For example, the processor 100 may control various functions of the electronic device 10 by executing programs stored in the memory 500.

The RAM 200 may temporarily store programs, data, or instructions. For example, programs and/or data stored in the memory 500 may be temporarily loaded into the RAM 200 according to the control or booting code of the processor 100. The RAM 200 may be implemented using a memory such as a dynamic RAM (DRAM) or a static RAM (SRAM).

The input/output device 400 may receive input data from a user or from the outside, and output a data processing result of the electronic device 10. The input/output device 400 may be implemented using at least one of a touch screen panel, a keyboard, and various types of sensors. In an embodiment, the input/output device 400 may collect information around the electronic device 10. For example, the input/output device 400 may include at least one of various types of sensing devices such as an imaging device, an image sensor, a light detection and ranging (LiDAR) sensor, an ultrasonic sensor, and an infrared sensor, or may receive a sensing signal from the device. In an embodiment, the input/output device 400 may sense or receive an image signal from the outside of the electronic device 10, and may convert the sensed or received image signal into image data, i.e., an image frame. The input/output device 400 may store the image frame in the memory 500 or provide the image frame to the machine learning processor 300.

The memory 500 is a storage location for storing data, and may store, for example, an operating system (OS), various programs, and various data. The memory 500 may be a DRAM, but is not limited thereto. The memory 500 may include at least one of a volatile memory and a non-volatile memory. The non-volatile memory may include a read only memory (ROM), a programmable ROM (PROM), an erasable programmable ROM (EPROM), an electrically erasable programmable ROM (EEPROM), a flash memory, a phase-change RAM (PRAM), a magnetic RAM (MRAM), a resistive RAM (RRAM), a ferroelectric RAM (FRAM), etc. The volatile memory may include a DRAM, an SRAM, a synchronous DRAM (SDRAM), etc. In addition, in an embodiment, the memory 150 may be implemented as a storage device such as a hard disk drive (HDD), a solid-state drive (SSD), a compact flash (CF), a secure digital (SD), a micro secure digital (Micro-SD), a Mini-SD, an extreme digital (xD), or a Memory Stick.

The machine learning processor 300 may train at least one of various types of machine learning models based on previously acquired training data, and may perform calculations based on the trained model. For example, the machine learning processor 300 may perform calculations based on received input data to generate inference values as calculation results, or retrain the machine learning model.

The types of machine learning models trained and inferred by the machine learning processor 300 may include supervised learning models, unsupervised learning models, and reinforcement learning models, and various types of models may be ensembled.

In addition, the machine learning processor 300 may generate a neural network model, train or learn a neural network, perform calculations based on received input data and generate information signals based on calculation results, or retrain a neural network. The neural network may include various types of neural network models such as a convolution neural network (CNN), a region with convolution neural network (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzmann machine (RBM), a fully convolutional network, a long short-term memory (LSTM) network, a classification network, etc., but is not limited thereto.

The electronic device 10 according to the embodiment of the present disclosure may perform preprocessing on input data by the processor 100. Preprocessing of the input data may be a process for effectively processing the collected data according to the purpose. For example, the processor 100 may perform data cleaning, data transformation, data filtering, data integration, and data reduction on medical images.

The processor 100 of the electronic device 10 according to the embodiment of the present disclosure compares a data amount that is processable in the resource with a data amount of medical information that needs to be processed, and the machine learning processor 300 may specify medical information that needs to be processed first among medical information based on a calculated comparison value. The processor 100 may instruct the resource to display the specified medical information.

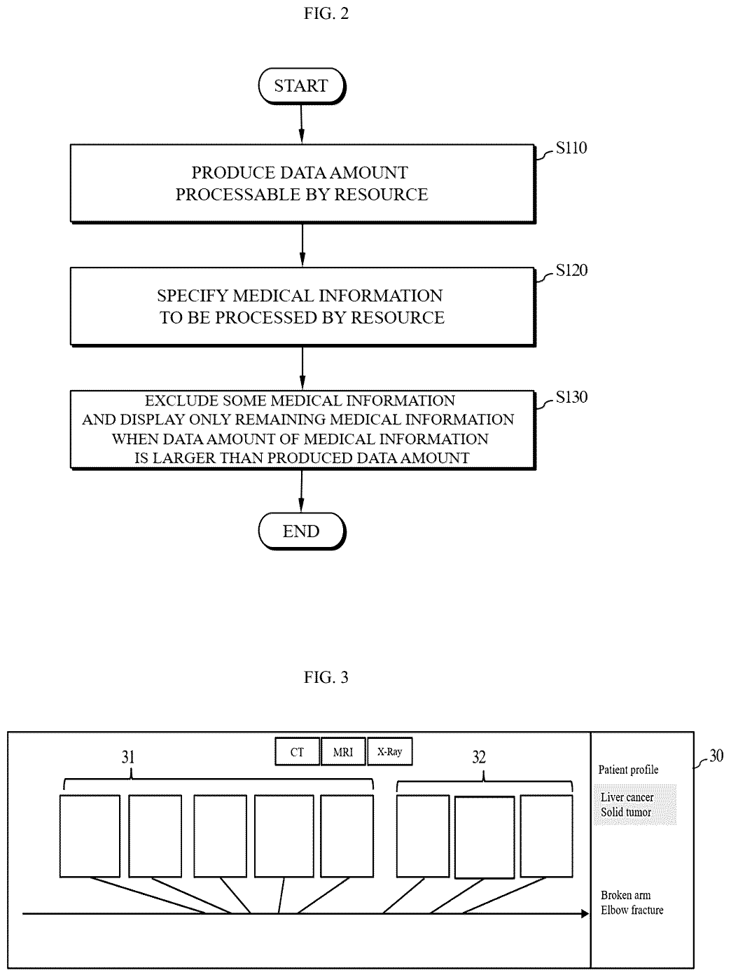

FIG. 2 is a flowchart showing a method of displaying medical information according to an embodiment.

Referring to FIG. 2, the electronic device 10 of the present disclosure may display medical information based on an allocated resource. The electronic device 10 may specify the status of the resource for which display of medical information is requested, and may exclude some medical information from the medical information and display the remaining medical information based on the specified status of the resource. For example, the electronic device 10 may classify the medical information into a plurality of groups according to importance, and may transmit only some medical information with a high priority among the classified groups to the resource.

In operation S110, the electronic device 10 may calculate a data amount that is processable by the resource. The electronic device 10 may identify a network status and performance of a processor of the resource. For example, the electronic device 10 may calculate a data amount that is receivable by the resource during a predetermined time period. In addition, the electronic device 10 may calculate a data amount that is processable by the resource during a predetermined time period.

In operation S120, the electronic device 10 may specify medical information to be processed by the resource according to a viewing mode requested by the user. The viewing mode may be specified according to a process range accessible by the resource. For example, the viewing mode may be divided into an editing mode in which the medical information is allowed to be edited by the user and a read-only mode in which editing access by the user is blocked.

According to an embodiment, when the viewing mode requested by the user is an editing mode, the electronic device 10 may include data of the medical information including modeling data of the medical information to be displayed and editing tool data associated with the modeling data. The editing tool data may include command data that allows the medical information to be rendered on the resource based on the modeling data, and rendered graphical user interface (GUI) information and a character user interface (CUI) to be edited.

According to an embodiment, when the viewing mode requested by the user is a read-only mode, the electronic device 10 may include data of the medical information including rendering data of the medical information to be displayed. The rendering data may be image data that has been rendered by the electronic device 10 based on modeling data of the medical information.

When the viewing mode is an editing mode, the electronic device 10 may transmit the modeling data and the editing tool data associated with the medical information, and the resource may directly perform rendering based on the modeling data to display the medical information.

In contrast, when the viewing mode is a read-only mode, the electronic device 10 may transmit a result generated by performing rendering based on the modeling data to the resource as image data. Accordingly, the resource may display the medical information by receiving only the image data to be displayed without directly performing rendering in the read-only mode.

In operation S130, the electronic device 10 may compare the data amount that is processable by the resource with the data amount of the medical information, and when the data amount of the medical information is larger, exclude some of the medical information and display only the remaining medical information. That is, the electronic device 10 may, upon identifying that the data amount of medical information to be processed by the resource is less than the data amount that is processable by the resource, transmit all of the medical information specified in operation S120 to the resource.

According to an embodiment, when the viewing mode is an editing mode, a data amount calculated to be processable by the resource based on the network status and the performance of the processor may be compared with a data amount of medical information. When the viewing mode is an editing mode, the electronic device 10 may specify medical information based on modeling data and editing tool data, and may compare a data amount that is receivable within a predetermined time based on the network status with a data amount of the specified medical information. The electronic device 10 may, upon identifying that the data amount of the specified medical information is greater than the data amount that is receivable within a predetermined time, exclude some information from the specified medical information and transmit only the remaining medical information to the resource.

The electronic device 10 may, upon identifying that the performance of the processor of the resource is insufficient to process the specified medical information, even after identifying that the data amount of the specified medical information is smaller than the data amount that is receivable during the predetermined time, exclude some information from the specified medical information and transmit only remaining medical information to the resource. For example, the electronic device 10 may calculate a computational speed of the processor required to render the medical information by modeling data. The electronic device 10 may calculate a data amount required in a process of rendering the modeling data, and may calculate a computational speed for processing the calculated data amount during a predetermined time.

In this case, the electronic device 10 may calculate the data amount required in the process of rendering the modeling data based on a ratio of GUI data and CUI data to image data among the modeling data. The image data may be original medical image data captured or acquired by an external server. The image data may be original data that may not be edited by an editing tool among all the data, and may be data that does not require rendering. In contrast, the GUI data and the CUI data may be data that may be customized by the user, and may be data that requires rendering. That is, the electronic device 10 may identify that a larger data amount is required in the process of rendering the modeling data when the ratio of GUI data and CUI data in the modeling data is high, even when the specified data amount is the same.

According to an embodiment, the electronic device 10 may divide the medical information into a plurality of groups according to the data amount of the medical information and the network status of the resource, and may transmit the divided medical information to the resource according to the priority. The electronic device 10 may divide an original medical image included in the medical information into a plurality of parts based on the network status of the resource. For example, when the data amount of the original medical image is 30 times the data amount that is receivable during a predetermined time, the electronic device 10 may divide the original medical image into more than 30 parts.

The electronic device 10 may transmit only some of a plurality of segmented medical images to the resource according to the priority. The electronic device 10 may, when transmitting a list of medical images similar to a target medical image to the resource, segment the similar medical images into a plurality of egmented medical images, extract a segmented medical image similar to the target medical image, and transmit the extracted segmented medical image to the resource.

That is, when it is identified that the network status of the resource is insufficient to transmit all of the plurality of original medical images, the electronic device 10 may reduce the number of original medical images to be transmitted, or may transmit only a part of the original medical image corresponding to the target medical image to the resource without reducing the number of medical images to be transmitted.

FIG. 3 is a drawing illustrating a storyboard 30 in which medical information is listed in time series according to an embodiment.

Referring to FIG. 3, when displaying a storyboard 30 in which medical information for a specified patient is listed in time series, the electronic device 10 may determine a time range of the storyboard 30 filtered according to a difference between a data amount that is processable by the resource and a data amount corresponding to the storyboard 30.

For example, upon identifying that only three pieces of medical information among a plurality of pieces of medical information are receivable and processed by the resource during a designated time in the storyboard 30 in chronological order, the electronic device 10 may divide the plurality of pieces of medical information into groups of three pieces of information in the order of the most recent medical information and transmit the divided medical information to the resource.

According to another embodiment, when there is medical information about a plurality of body parts, the electronic device 10 may divide the medical information by body part. For example, when a patient has undergone treatment for “liver cancer” and treatment for “broken arm” and information for each treatment is displayed in chronological order, the electronic device 10 may divide medical information 31 related to liver cancer and medical information 32 related to the broken arm. When one body part is selected by the resource, the medical information related to the selected body part among the divided medical information may be preferentially displayed.

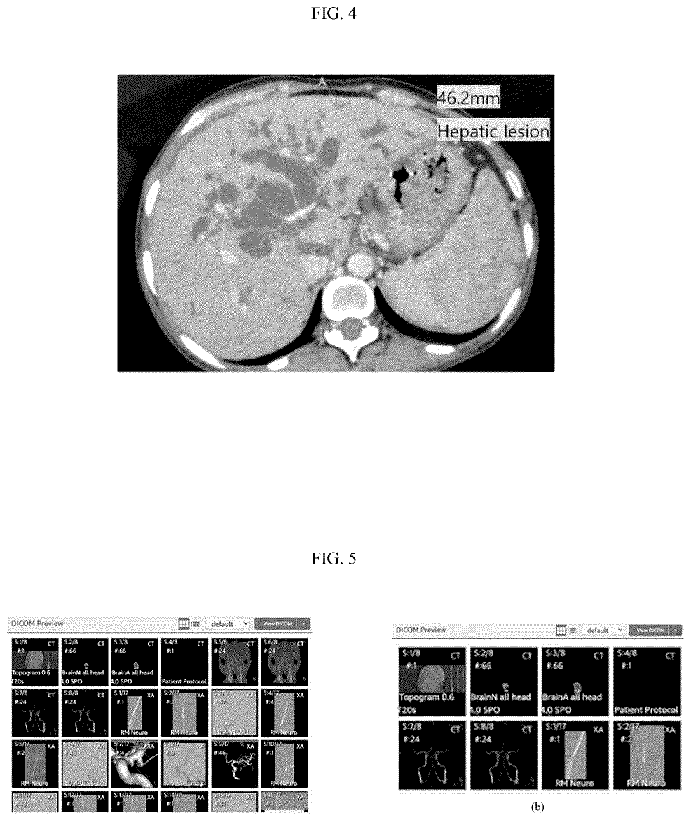

FIG. 4 is a drawing illustrating a medical image on which annotation information is written according to an embodiment.

Referring to FIG. 4, when the medical information is an image including annotation information or a measurement value, the electronic device 10 may display a medical image excluding the annotation information or the measurement value as a highest priority.

For example, the medical information may be CT image data of a cross-section of the liver, and the electronic device 10 may specify and mark the location of a lesion identified in the image data and annotate the size and location of the lesion. That is, the electronic device 10 may generate description information of an original medical image as CUI data or GUI data, and may generate the original medical image and the CUI/GUI data as medical information.

In this case, when the viewing mode requested by the user is a read-only mode, the electronic device 10 may generate rendering data by adding the CUI/GUI data to the original medical image. The rendering data may be data in which the GUI and CUI are imaged together with the original medical image to prevent editing. Accordingly, when the viewing mode is a read-only mode, the electronic device 10 may provide only the minimum data required for the user to the resource without including modeling data and editing tool data.

In contrast, when the viewing mode requested by the user is an editing mode, the electronic device 10 may include data of the medical information including modeling data of the medical information to be displayed and editing tool data associated with the modeling data. The modeling data may include the original medical image and the CUI/GUI data. The resource may receive the modeling data and the editing tool data as the medical information, and may perform direct rendering based on the modeling data and the editing tool data.

According to an embodiment, when the viewing mode is an editing mode, the electronic device 10 may determine whether to transmit all the medical information to the resource based on the network status of the resource and the performance of the processor, and may determine to transmit only a portion of the medical information to the resource. In this case, even when the viewing mode is an editing mode, the electronic device 10 may transmit the rendering data on which rendering has been completed to the resource instead of the modeling data and the editing tool data.

That is, the electronic device 10 may, upon identifying that the data amount that is processable by the resource is significantly small compared to the entire data amount of the medical information, block the editing of the medical information even in the viewing mode in which editing of the medical information is requested.

FIG. 5 is a drawing illustrating an example in which medical information is displayed on a different resource according to an embodiment.

Referring to FIG. 5, the electronic device 10 may preferentially transmit only some information of all the medical information to the resource based on not only the data amount that is processable by the resource but also the size of the screen on which the medical information is displayed and the type of the resource. The resource may transmit the type of the resource and the screen size to the electronic device 10, and the electronic device 10 may determine the range of the medical information to be transmitted to the resource based on the type of the resource and the screen size.

For example, the resource may include a PC device and a smartphone terminal device, and the range of the medical information to be transmitted may be determined based on whether the medical information is displayed by a monitor connected to the PC device or by a screen of the smartphone terminal device.

The electronic device 10 may transmit medical images associated with a target medical image as medical information to the resource, and may determine the number of medical images to be transmitted based on the type and screen size of the resource.

According to an embodiment, each of the medical images may have text metadata related to the medical image characteristics mapped thereto. The electronic device 10 may designate the text metadata to one of a plurality of layers. For example, text metadata of an affected area location may be mapped to one of hierarchized body tissue locations.

When transmitting only some of the medical images associated with the target medical image to the resource, the electronic device 10 may preferentially transmit the medical images of which text metadata matches that of the target medical image at the lowest layer to the resource. Thereafter, the electronic device 10 may, after transmitting all the medical images with matching text metadata at the lowest layer, transmit medical images of which text metadata matches that of the target medical image at an upper layer of the lowest level to the resource.

Accordingly, the electronic device 10 may select medical information required for the user with priority and efficiently provide medical information in a situation in which data processing is limited.

Meanwhile, the methods according to the various embodiments of the present invention described above may be implemented in the form of an application or software program that may be installed on an existing electronic device.

In addition, the entirety or part of the method may be configured as several software function modules and implemented in an OS. Alternatively, each operation may be configured as one software function module, or each operation may be combined to be configured as one software function module and implemented on an OS. Accordingly, even when some embodiments of the present disclosure are not implemented entirely as one software function module, when several software function modules implement each operation of the present disclosure and several software function modules are implemented in one OS, it should be understood that the method of the present disclosure has been implemented.

In addition, the methods according to the various embodiments of the present invention described above may be implemented only with a software upgrade or a hardware upgrade for an existing electronic device. In addition, the various embodiments of the present invention described above may also be performed through an embedded server equipped in the electronic device, or an external server of the electronic device.

Meanwhile, according to an embodiment of the present invention, the various embodiments described above may be implemented using software, hardware or a combination thereof, as software including instructions stored in a computer readable recording medium that can be read by a computer or a similar device. In some cases, the embodiments described in this specification may be implemented by the processor itself. According to a software implementation, embodiments such as the procedures and functions described in this specification may be implemented as separate software modules. Each of the software modules may perform one or more functions and operations described in this specification.

Meanwhile, a computer or a similar device may be a device capable of invoking instructions stored from a storage medium and operating in accordance with the invoked instructions and may include a device according to the disclosed embodiments. When the instructions are executed by a processor, the processor may directly perform a function corresponding to the instructions or may use other components to perform the function under the control of the processor. The constructions may include code generated or executed by a compiler or an interpreter.

A recording medium that is read by a machine may be provided in the form of a non-transitory computer readable recording medium. Here, when a recording medium is referred to as “non-transitory,” it can be understood that the recording medium is tangible and does not include a signal, but rather that data is semi-permanently or temporarily stored in the recording medium. A non-transitory computer readable medium is a medium that can store data semi-permanently and can be read by a device, rather than a medium that stores data for a short period of time, such as a register, cache, or memory. Examples of non-transitory computer readable media may include a compact disc (CD), a digital versatile disc (DVD), a hard disk, a Blu-ray disc, a Universal Serial Bus (USB), a memory card, a read-only memory (ROM).

As is apparent from the above, a method of displaying medical information according to an exemplary embodiment of the present disclosure can differentially display medical information based on resources of a terminal requesting display of medical information. Accordingly, a user can rapidly obtain the required medical information by an optimized method.

The effects of the present disclosure are not limited to the effects described above, and other effects that are not described will be clearly derived and understood by those skilled in the art from the following description. That is, unintended effects resulting from implementing the exemplary embodiments of the present disclosure may also be derived from the exemplary embodiments of the present disclosure by a person having ordinary skill in the art.

The present invention has been described with reference to exemplary embodiments illustrated in the drawings and specification. Although the embodiments have been described using specific terms in the specification, the embodiments disclosed above should be construed as being illustrative rather than limiting the present invention, and those skilled in the art should appreciate that various substitutions, modifications, and changes are possible without departing from the scope and spirit of the present invention. Therefore, the scope of the present invention is defined by the appended claims of the present invention.

What is claimed is:

1. A method of displaying medical information based on a resource allocated to a medical information display, the method comprising:
producing a data amount that is processable by the resource;
specifying medical information to be processed by the resource according to a viewing mode requested by a user; and
when a data amount of the specified medical information is larger than the produced data amount, excluding some information from the medical information and displaying the remaining medical information.

2. The method of claim 1, wherein the viewing mode is divided into an editing mode in which the medical information is editable by the user and a read-only mode in which editing access by the user is blocked, and the data amount of the specified medical information increases as a range of processes that are processed according to the viewing mode increases.

3. The method of claim 2, wherein the displaying includes
when the viewing mode is an editing mode, comparing a data amount produced based on a network status of the resource and performance of a processor in the produced data amount with a data amount of medical information; and
when the viewing mode is a read-only mode, comparing a data amount produced based on a network status of the resource in the produced data amount with the data amount of the medical information.

4. The method of claim 1, wherein the displaying includes
when the medical information is generated as a storyboard listed in time series, determining a time range of a storyboard filtered based on a difference between the data amount of the medical information and the produced data amount.

5. The method of claim 1, wherein the displaying includes
when the medical information is an image including annotation information or a measurement value, displaying an image excluding the annotation information or the measurement value as a highest priority.

6. The method of claim 1, wherein the specifying of the medical information includes segmenting the medical information based on a data amount of the medical information and a network status of the resource; and
transmitting the segmented medical information to the resource based on a priority of the segmented medical information.

7. The method of claim 6, wherein the segmenting of the medical information includes:
when a display of related medical information associated with target medical information is requested by a user, classifying the related medical information into a plurality of groups; and
identifying a priority between the classified groups.
